(19) United States
(12) Patent Application Publication
(10) Pub. No.: US 2014/0255517 A1
(43) Pub. Date: Sep. 11, 2014

(54) MANAGING POST-TRAUMATIC STRESS DISORDER

(71) Applicants: Kerry J. Ressler, Atlanta, GA (US); Paul J. Marvar, Washington, DC (US)

(72) Inventors: Kerry J. Ressler, Atlanta, GA (US); Paul J. Marvar, Washington, DC (US)

(73) Assignee: EMORY UNIVERSITY, Atlanta, GA (US)

(21) Appl. No.: 14/197,399

(22) Filed: Mar. 5, 2014

Related U.S. Application Data
(60) Provisional application No. 61/773,220, filed on Mar. 6, 2013.

(51) Int. Cl. A61K 31/4178; A61K 45/06

(52) U.S. Cl. CPC A61K 31/4178; A61K 45/06
USPC 424/715; 514/311; 514/381; 514/213.01; 514/91; 514/321; 514/252.17; 514/171; 514/217; 514/253.04; 514/254.05; 514/300; 514/242; 514/252.15; 514/325; 514/259.41

(57) ABSTRACT

In certain embodiments, the disclosure relates to methods of treating or preventing posttraumatic stress disorder comprising administering a pharmaceutical composition comprising effective amount of an angiotensin-converting enzyme inhibitor to a subject in need thereof. In certain embodiments, the angiotensin-converting enzyme inhibitor is administered in combination with an angiotensin receptor blocker.

### CROSS REFERENCE TO RELATED APPLICATIONS

This application claims priority to U.S. Provisional Application No. 61/773,220 filed Mar. 6, 2013, hereby incorporated by reference in its entirety.

### STATEMENT REGARDING FEDERALLY FUNDED RESEARCH

This invention was made with government support under Grant K99 HL107675-01 awarded by the National Institutes of Health. The government has certain rights in the invention.

### BACKGROUND

Posttraumatic stress disorder (PTSD) is a debilitating, stress-related psychiatric illness associated with trauma exposure. While the lifetime prevalence of PTSD in the general population is estimated to be 5%-10%, the prevalence of PTSD in low-income, urban, primary care patients has been estimated to be as high as 45%. Higher still is the prevalence of lifetime trauma exposure within this population, approximately 88%. Modest benefits have been seen from early access to cognitive behavioral therapy, as well as from some medications such as propranolol. However, there is still a need for improved therapies.

Chronic stress, involving exposure to frequent and early traumatic events, has been implicated in multiple adverse health outcomes including cardiovascular-associated diseases, such as hypertension. Kibler reported posttraumatic stress is associated with cardiovascular risk and cardiovascular disease. See J Trauma Dissociation, 2009, 10(2):135-150. See also Buckley & Kaloupek, Psychosom Med, 2001, 63(4):585-594 and Pole, Psychol Bull, 2007,133(5):725-746.

An approach for the treatment of hypertension involves the pharmacologic inhibition of the renin-angiotensin system (RAS) by angiotensin-converting enzyme (ACE) inhibitors and angiotensin receptor blockers (ARBs). Angiotensin converting enzyme inhibitors prevent the synthesis of angiotensin II, while ARBs block interaction between angiotensin II and its receptor. Benicky et al. report angiotensin II AT(1) receptor blockade ameliorates brain inflammation. See Neuropsychopharmacology, 2011, 36 (4):857-870. Anderson reports potential neuroprotective benefits of angiotensin receptor blockers. See Hypertens, 2010, 28(3):429. Chen et al. report blood pressure reactivity to emotional stress is reduced in AT1A-receptor knockout mice. Hypertens Res, 2009, 32(7):559-64. Krause et al. report that circulating blood-borne angiotensin II in response to stress acts on the AT1 receptor in the hypothalamus. J Neurosci., 2011, 31(42):15009-15. Khoury et al., report that angiotensin-converting enzyme inhibitors and angiotensin receptor blockers are associated with fewer traumatic stress symptoms. See J Clin Psychiatry, 2012, 73:849-855. See also Li et al., BMJ, 2010, 340:b5465, Saxby et al., Neurology, 2008, 70(19, pt 2):1858-1866. Zanchetti et al., Blood Press, 2006, 15(2):71-79, and Weber, Manag Care Interface, 2005,18(2): 47-54. References cited herein are not an admission of prior art.

### SUMMARY

In certain embodiments, the disclosure relates to methods of treating or preventing posttraumatic stress disorder comprising administering a pharmaceutical composition comprising effective amount of an angiotensin-converting enzyme inhibitor to a subject in need thereof. In certain embodiments, the angiotensin-converting enzyme inhibitor is administered in combination with an angiotensin receptor blocker. It is not intended that embodiments of this disclosure be limited by any particular mechanism; however, it is also contemplated that the angiotensin receptor blockers may work by themselves without the angiotensin-converting enzyme inhibitor, and the disclosure contemplates administration alone or in combination.

In certain embodiments, the disclosure relates to methods of treating or preventing posttraumatic stress disorder comprising administering a pharmaceutical composition comprising effective amount of an angiotensin receptor blocker to a subject in need thereof. In certain embodiments, the angiotensin receptor blocker is administered in combination with an angiotensin-converting enzyme inhibitor.

In certain embodiments, the disclosure relates to methods of reducing fear memory comprising administering a pharmaceutical composition comprising effective amount of an angiotensin-converting enzyme inhibitor to a subject in need thereof. In certain embodiments, the angiotensin-converting enzyme inhibitor is administered in combination with an angiotensin receptor blocker.

In certain embodiments, the disclosure relates to methods of treating or preventing reducing fear memory comprising administering a pharmaceutical composition comprising effective amount of an angiotensin receptor blocker to a subject in need thereof. In certain embodiments, the angiotensin receptor blocker is administered in combination with an angiotensin-converting enzyme inhibitor.

In certain embodiments, the subject is at risk of, exhibiting symptoms of, or diagnosed with posttraumatic stress disorder. In certain embodiments, the subject experienced military combat.

In certain embodiments, the angiotensin-converting enzyme inhibitor is selected from perindopril, captopril, enalapril, eprosartan, lisinopril, quinapril, benazepril, imidapril, trandolapril, fosinopril, ramipril, and zofenopril.

In certain embodiments, the angiotensin receptor blocker is losartan, irbesartan, olmesartan, candesartan, valsartan, telmisartan, and azilsartan.

In certain embodiments, the pharmaceutical composition is administered in combination with another therapeutic agent.

In certain embodiments, the therapeutic agent is selected from propranolol, citalopram, escitalopram, fluoxetine, fluvoxamine, paroxetine, sertraline, prazosin, clonidine, carbamazepine, topiramate, zolpidem, lamotrigine, valproic acid, lithium carbonate, buspirone, risperidone, cyproheptadine, nefazodone, trazodone, amitriptyline, imipramine, phenelzine, or corticosterone, and combinations thereof.

In certain embodiments, the disclosure relates to methods of treating or preventing posttraumatic stress disorder comprising administering a pharmaceutical composition comprising an effective amount of an angiotensin receptor blocker to a subject in need thereof.

In certain embodiments, the subject is at risk of, exhibiting symptoms of, or diagnosed with posttraumatic stress disorder.

In certain embodiments, the angiotensin receptor blocker is losartan, irbesartan, olmesartan, candesartan, valsartan, telmisartan, and azilsartan.

In certain embodiments, the pharmaceutical composition is administered in combination with another therapeutic agent.

In certain embodiments, the therapeutic agent is selected from propranolol, citalopram, escitalopram, fluoxetine, fluvoxamine, paroxetine, sertraline, prazosin, clonidine, carbamazepine, topiramate, zolpidem, lamotrigine, valproic acid, lithium carbonate, buspirone, risperidone, cyproheptadine, nefazodone, trazodone, amitriptyline, imipramine, phenelzine, or corticosterone and combinations thereof.

### BRIEF DESCRIPTION OF THE FIGURES

FIG. 1 shows recruitment and selection of participants in a study.

FIG. 2 shows a table of the descriptive overview of variables stratified by PTSD diagnosis (N=505). Abbreviations: ACE=angiotensin-converting enzyme, ARB=angiotensin receptor blocker, BMI=body mass index, CTQ=childhood trauma questionnaire, GED=General Educational Development test, PTSD=posttraumatic stress disorder, TEI=traumatic events inventory

FIG. 3 shows a table of potential confounders, stratified by ACE Inhibitor or ARB (N=505). Abbreviations: ACE=angiotensin-converting enzyme, ARB=angiotensin receptor blocker, BMI=body mass index, CTQ=childhood trauma questionnaire, GED=General Educational Development test, TEI=traumatic events inventory.

FIG. 4 shows a table of univariable analysis of variance of PTSD symptoms by PSS total score and by symptom subtype. Abbreviations: ACE=angiotensin-converting enzyme, ARB=angiotensin receptor blocker, PSS=modified PTSD Symptom Scale, PTSD=posttraumatic stress disorder.

FIG. 5 shows a table multi-variable linear regression of PSS and CAPS Score ^{a}Adjusted for childhood trauma, adult trauma, age, sex, employment, and β-blocker. R2=0.29, F=27.20. ^{b}2 Adjusted for childhood trauma, adult trauma, age, diuretics, employment and β-blocker. R2=0.22, F=18.20. ^{c}Adjusted for childhood trauma, adult trauma, age, other blood pressure medications, β-blockers, and income. R2=0.27, F=16.20. ^{d}Adjusted for childhood trauma, adult trauma, age, sex, and β-blockers. R−=0.22, F=21.17. ^{e}ACE inhibitor or ARB. ^{f}Adjusted for childhood trauma, adult trauma, age, diuretics, employment, and β-blockers. R²=0.22, F=18.20. ^{g}Adjusted for childhood trauma, adult trauma, and employment. R²=0.190, F=22.07. Abbreviations: ACE=angiotensin-converting enzyme, ARB=angiotensin receptor blocker, CAPS=Clinician Administered PTSD Scale, PSS=modified PTSD Symptom Scale, PTSD=posttraumatic stress disorder.

FIG. 6 shows data indicating that angiotensin type 1 (AT1R) receptor inhibition enhances the extinction of learned fear: Prior to losartan treatment (pre-drug) acquired fear during cued fear conditioning with five tone-shock pairings in pre-vehicle (n=10) and pre-losartan (n=11) groups (A). 24 hr following fear acquisition, losartan (1 mg/kg or 10 mg/kg i.p) was given prior to extinction training (B, E; n=20-25/group). 24 hr following extinction training, and in the absence of drug, mice were tested for extinction retention of learned fear, expressed as total average freezing and in blocks of 5 CS tones (C,D,F,G).

FIG. 7 shows data indicating that acute administration of losartan (10 mg/kg, i.p.) does not affect baseline blood pressure or anxiety measures: Blood pressure measured by radiotelemetry following acute administration of saline (n=4) or losartan (10 mg/kg, i.p n=4) (A). Distance traveled during open-field testing (B) and percent time in open arms of elevated plus maze test of vehicle (C) (n=6-8/group).

FIG. 8 shows data indicating chronic inhibition of angiotensin type 1 (AT1R) receptor reduces fear expression and enhances extinction of learned fear: Following two weeks of losartan (10 mg/kg/day) (n=17) or vehicle (n=13) infusion, fear acquisition expressed as percent freezing during cued fear conditioning with five tone-shock pairings (A). Extinction training/fear expression was tested and total average freezing within the session (B) and represented in blocks of 5 CS tones (C) 24 hr following fear acquisition. Extinction retention of learned fear, expressed as total average freezing and in blocks of 5 CS tones are shown in panels (D) and (E) (n=18-20).

FIG. 9 shows data indicating the effect of chronic losartan on central and peripheral indices of stress activation: Plasma corticosterone levels (A) and plasma renin activity (B) in mice treated with vehicle or losartan for 2 weeks following acute restraint stress (n=9-10/group). Induction of c-Fos protein quantified as number of positive cell counts per section of paraventricular nucleus (C) (n=5-7/group). Representative images showing reduced cFos protein induction in losartan versus vehicle animals exposed to acute restraint stress (D-E).

FIG. 10 shows data indicating reduced AT₁R amygdala mRNA expression following extinction training in losartan treated mice: AT₁R messenger RNA (mRNA) levels in the amygdala of vehicle (n=7) or losartan infused mice (n=7) (A). Coronal brain section with black circles designating the surrounding regions of the amygdala isolated for reverse transcriptase quantitative polymerase chain reaction.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

Prior to describing the various embodiments, the following definitions are provided and should be used unless otherwise indicated.

“Subject” refers any animal, preferably a human patient, livestock, rodent, monkey or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

### Posttraumatic Stress Disorder (PTSD)

Posttraumatic Stress Disorder (PTSD) is a debilitating condition that follows a terrifying event, e.g., the person has been exposed to a traumatic event in which the person experienced, witnessed, or was confronted with an event or events that involved actual or threatened death or serious injury, or a threat to the physical integrity of self or others and the response of the person involved intense fear, helplessness, or horror.

The traumatic event typically is persistently re-experienced in one or more of the following ways: recurrent and intrusive distressing recollections of the event, including images, thoughts, or perceptions; recurrent distressing dreams of the event; acting or feeling as if the traumatic event were recurring (includes a sense of reliving the experience, illusions, hallucinations, and dissociative flashback episodes, including those that occur on awakening or when intoxicated). The person has intense psychological distress at exposure to internal or external cues that symbolize or resemble an aspect of the traumatic event; and physiological reactivity on exposure to internal or external cues that symbolize or resemble an aspect of the traumatic event.

The individual typically also has persistent avoidance of stimuli associated with the trauma and numbing of general responsiveness (not present before the trauma), as indicated by 3 or more of the following: efforts to avoid thoughts, feelings, or conversations associated with the trauma; efforts to avoid activities, places, or people that arouse recollections of the trauma; inability to recall an important aspect of the trauma; significantly diminished interest or participation in significant activities; feeling of detachment or estrangement from others; restricted range of affect (e.g., unable to have loving feelings); sense of a foreshortened future (e.g., does not expect to have a career, marriage, children, or a normal life span).

The person typically has persistent symptoms of increased arousal (not present before the trauma), as indicated by 2 or more of the following: difficulty falling or staying asleep, irritability or outbursts of anger, difficulty concentrating, hypervigilance, and exaggerated startle response.

The person typically has the disturbance, which has lasted for at least a month, causes clinically significant distress or impairment in social, occupational, or other important areas of functioning.

### The Renin-Angiotensin Pathway in Post-Traumatic Stress Disorder

Results disclosed herein indicate that ACE inhibitors and ARB medications have protective effects on PTSD symptoms among individuals exposed to trauma. After adjustment, the effect of ACE inhibitors and ARBs on the reduction of PTSD symptoms remained significant, using both the PSS and the CAPS measurements (P=0.028 and P=0.010, respectively), thought to be a thorough measurement of current and lifetime PTSD symptoms.

In addition, this analysis indicates that ACE inhibitors and ARBs may preferentially affect the severity of hyperarousal and intrusive PTSD symptoms. Other medications that have been shown to decrease these symptoms include prazosin, clonidine, guanfacine, and propranolol, all of which target the noradrenergic system. However, results from this analysis did not demonstrate any interaction in individuals taking both 13-blockers and ACE inhibitors/ARBs on PTSD symptoms. In fact, while univariate associations demonstrated a significant effect of 13-blockers on hyperarousal symptoms, these effects did not remain after adjustment for confounders. This was somewhat surprising, given that propranolol has also been used both to ameliorate PTSD symptoms and, in some studies, to prevent PTSD.

In certain designs, the cross-sectional design makes causation difficult to evaluate. Notably, if collinearity between taking a blood pressure medication and stress-related hypertension were the case, one would expect greater PTSD symptom severity to associate with taking blood pressure medications. In contrast, with the ARB/ACE inhibitor class, an unexpected decrease in PTSD symptoms associated was found with medication use.

Experiments disclosed herein support a role for the renin-angiotensin pathway in stress-related disorders. Studies herein provide an analysis examining the effect of ACE inhibitors and ARB medications on PTSD symptoms in individuals exposed to trauma. Since ACE inhibitors and ARB medications are safe and widely used to treat hypertension, they may be are targets to consider for treatment and potential protection against PTSD symptoms.

### Effect of Angiotensin Type 1 Receptor Inhibition on Fear Memory

Patients with PTSD and other anxiety disorders are thought to have deficits in extinction of aversive memories. Similarly, rodents with anxiety-like behavior or trauma exposure demonstrate a deficit in extinction of conditioned fear. Studies herein indicate that animals treated with losartan using either a single dose or chronically for 2 weeks show less retention of fear memory or enhanced extinction as indicated by reduced freezing behavior. Interestingly, these effects were independent of any change in cardiovascular, anxiety or locomotor activities.

It is possible that AT1R antagonism could influence the level of fear acquired during fear training. However, at the doses we used, animals exposed to losartan for 2 weeks acquired fear similarly to control groups and are thus in line with other studies showing that losartan does not influence baseline anxiety levels in rodents when given chronically or acutely or on acquisition of an aversive memory. Moreover, these data demonstrate that AT1 R inhibition during fear conditioning enhances the extinction of an aversive memory and improves emotional learning, thus suggesting a role for endogenous angiotensin II in fear related neurobiological processes.

In most animal studies examining the role of angiotensin 11 and AT1 antagonism in learning and memory, inhibitory avoidance learning paradigms have been utilized, and these data have produced mixed results. For example, some have shown that angiotensin II improves avoidance memory, while others using similar learning paradigms have shown that angiotensin II impairs or has no effect on learning and memory. Given that one of the main aims of studies herein was to further understand the role of the renin-angiotensin II system in the fear response, Pavlovian fear conditioning, a robust animal model for assessing memory in fear learning in both animals and humans, was utilized Inhibition of the AT1 receptor plays a role in the extinction of fear memory. In comparing these data to studies using inhibitory avoidance, results herein suggest that angiotensin II improves memory because in the presence of the AT1 antagonist, memory retention as determined by freezing behavior was attenuated. On the other hand, these results are in contrast to some studies using inhibitory avoidance that show improvements in aversive memory following AT 1 antagonism.

These opposing results are likely due to study differences in aversive learning paradigms, the dose of the AT1 antagonist and/or whether the drug was delivered via brain injection or systemically and time of antagonist administration (i.e., prior to acquisition or retention).

Studies have also shown that avoidance learning paradigms have produced inconsistent results with regard to the involvement of brain structures essential to the fear response such as the amygdala.

The amygdala, part of the limbic system located in the temporal lobe of the brain, is an integral part of the fear circuitry and key inputs to the amygdala from the medial prefrontal cortex are thought to be required for the extinction of fear. Immunohistological studies have revealed that brain AT1 receptors are expressed throughout regions involved in emotional learning including the amygdala and hippocampus. Moreover, animal studies have demonstrated a role for brain angiotensin II acting via its AT1 receptor in the neuroendocrine and autonomic response to stress. In line with these data, animals in the studies herein treated with losartan for 14 days have less c-Fos activation in the paraventricular nucleus during restraint stress. Moreover, during extinction training losartan treated animals showed decreased amygdala AT1 receptor mRNA expression. These data may suggest that reduced AT1 receptor interaction may be contributing to the enhanced extinction in these mice. It suggests that stress-response feedback regulation of central brain regions are altered with angiotensin receptor blockade. The precise mechanism(s) for this enhanced extinction following AT1 inhibition is currently unknown and may involve other brain angiotensin receptor subtypes such as AT2 and AT4.

Brain angiotensin also interferes with different neurotransmitters and hormones such as norepinephrine, serotonin, vasopressin, and dopamine which are all involved in memory consolidation. Moreover, angiotensin II has been found to modulate neurotrophic factors such as brain derived neurotrophic factor (BDNF) a critical molecular mediator in learning and memory. In addition to BDNF, angiotensin II can activate multiple other signaling pathways that may influence AT1 receptor expression and function in fear memory. For example, Nostramao et al. report in vitro studies indicating that pituitary adenylate cyclase-activating polypeptide (PACAP), a peptide in the cellular stress response, modulates the angiotensin receptor. Interestingly, in a clinical study, a single nucleotide polymorphism in the PACAP receptor gene as well as differential levels of circulating PACAP peptide have recently been linked to level of PTSD severity. These studies demonstrate important links between the brain angiotensin system and PTSD.

In an animal model of PTSD it has been demonstrate that inhibition of the AT1 receptor enhances the extinction of fear memory. Importantly, this was shown following both acute and chronic administration of losartan and was independent of effects on blood pressure, measures of anxiety and fear acquisition. Furthermore, AT1 mRNA expression is reduced in losartan treated animals following extinction training, which implies that downstream AT1 signaling events may be important in consolidation of extinction of fear. Importantly, these data support use of this class of medications to treat or prevent PTSD.

### EXAMPLES

### Subjects, Sample Recruitment, and Results

Secondary analysis was examined data on 505 individuals, who were part of a larger cross-sectional study investigating the genetic and environmental factors that contribute to PTSD. From 2006 to November 2010, participants were recruited from the waiting rooms of primary care clinics, obstetric-gynecologic clinics, or the pharmacy at Grady Memorial Hospital (Atlanta, Ga.). One of the largest public hospitals in the United States, Grady serves a primarily African American and highly traumatized, low income, inner-city population. Recruitment took place Monday-Friday during regular clinic hours. Those subjects who agreed to participate completed a number of self-report measures, taking 45-75 minutes to complete.

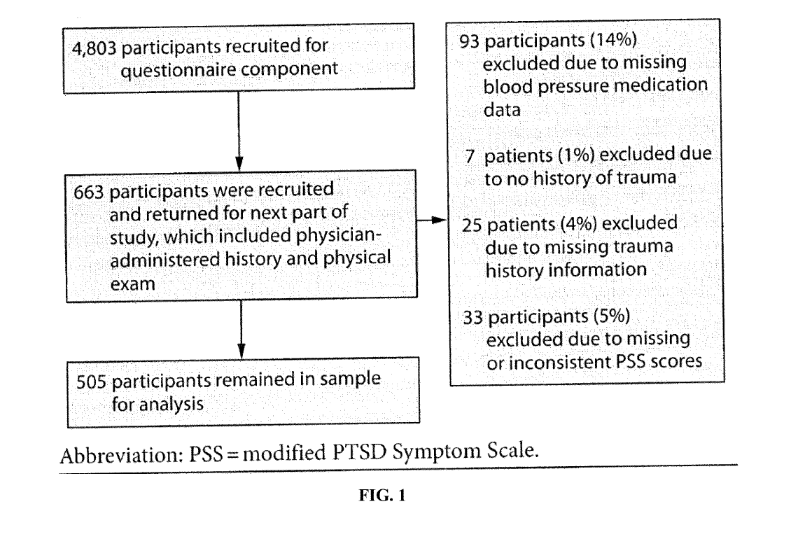

The primary exposure of interest was taking an ACE inhibitor or ARB; therefore, individuals whose information on blood pressure medications was missing were excluded from analysis (93 subjects, 14% of the sample). Patients who had missing information on the PTSD Symptom Scale (PSS) were also excluded from the analysis (n=28 or 4.9% of the remaining sample). To examine the association of ACE inhibitors or ARBs with PTSD symptoms among individuals exposed to traumatic events, only individuals who reported 1 or more traumatic events on the Childhood Trauma Questionnaire (CTQ) or Traumatic Event Inventory (TEI) were included in the analysis, leaving a sample of 505 individuals. A flowchart of the selection process for the sample is provided (FIG. 1).

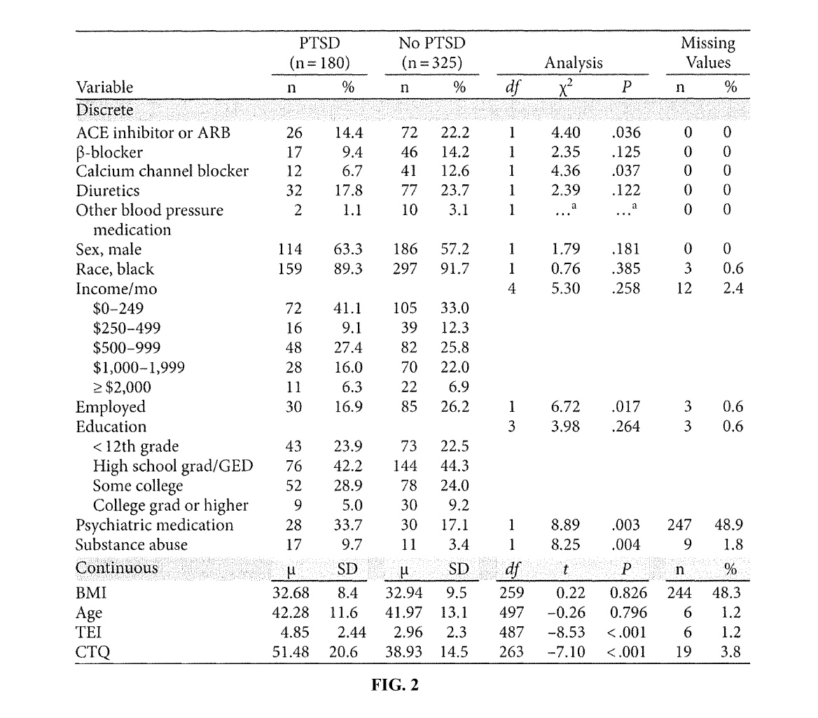

Among the 505 individuals exposed to at least 1 traumatic event, 180 met criteria for PTSD diagnosis based on PSS score. In the sample, 98 individuals were taking ACE inhibitors or ARBs, 63 were taking 13-blockers, 53 were taking CCBs, 109 were taking diuretics, and 12 were taking other blood pressure medications. A significant univariate association was found between PTSD diagnosis and ACE inhibitor or ARB status (FIG. 2).

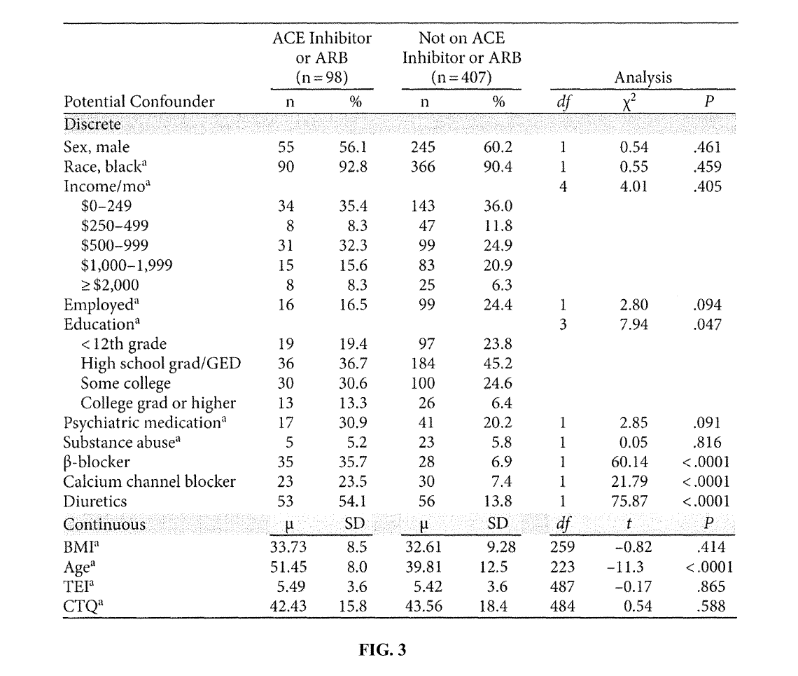

Of 98 individuals taking an ACE inhibitor or ARB, 26 met criteria for PTSD diagnosis using PSS; of 407 individuals not taking an ACE inhibitor or ARB, 154 met criteria for PTSD diagnosis (X² t value=4.40, P=0.036). Covariates demonstrating significant differences based on PTSD diagnosis included taking a CCB, employment, current psychiatric medication, current substance abuse, total adult trauma experienced, and childhood trauma (FIG. 2). Significantly different potential confounders stratified by ACE inhibitor or ARB included age, education, and treatment with β-blockers, CCBs, and diuretics (FIG. 3).

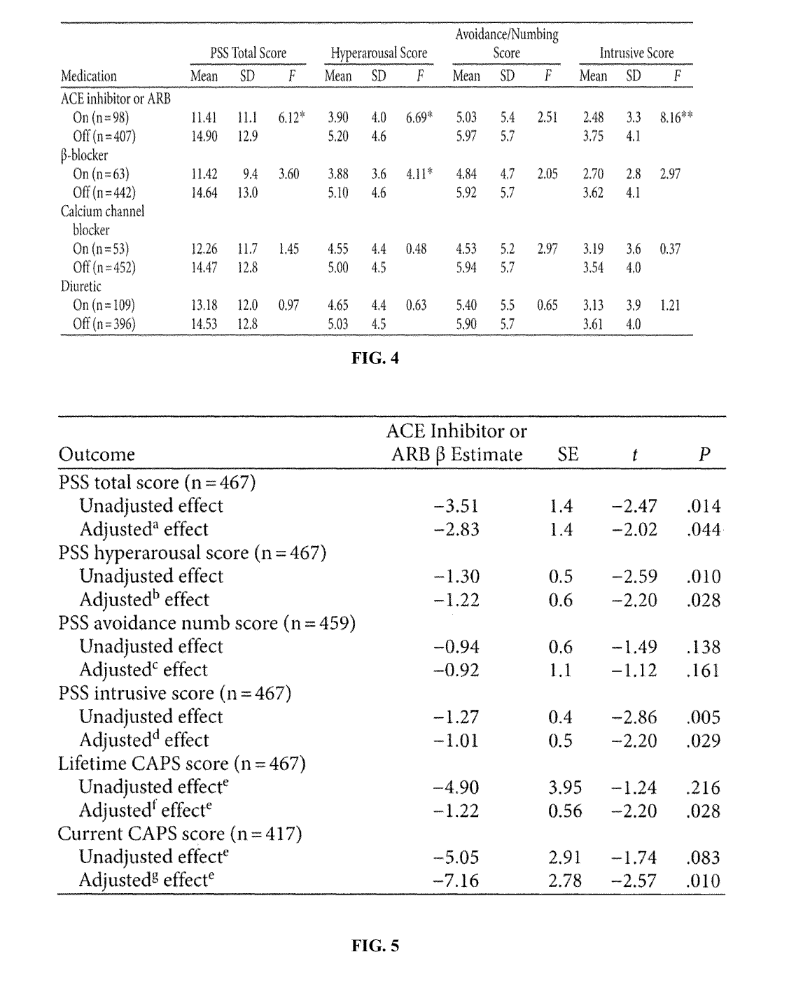

Mean PSS scores (total and by subtype) for individuals receiving different blood pressure medications are shown in FIG. 4. A significant difference in mean (SD) total PSS score was only found based on ACE inhibitor or ARB status (11.41±11.1 for ACE inhibitor/ARB treated and (14.90±12.9) for non-ACE inhibitor or ARB treated, F=6.12, P=0.014). When examined by PTSD subtype, individuals taking ACE inhibitors or ARBs and/or β-blockers demonstrated significant differences in mean (SD) hyperarousal score (3.90±4.0 and 5.20±4.6 on and off ACE inhibitors or ARBs, respectively; 3.88±3.6 and 5.10±4.6 on and off β blockers, respectively). No significant differences in mean avoidance/numbing score were found for any blood pressure medications. Lastly, significant differences in mean (SD) intrusive thoughts score were limited to comparisons of individuals taking versus not taking ACE inhibitors or ARBs (2.48±3.3 for ACE inhibitor/ARB treated and 3.75±4.1 for non-ACE inhibitor/ARB treated).

Given the comorbidity of depression with PTSD, the effects of ACE inhibitor I ARB status on depressive symptoms were also examined. In the analyzed traumatized sample, individuals taking ACE inhibitors/ARBs were found to have lower total BDI scores than individuals not taking ACE inhibitors/ARBs, but the results were not statistically significant (13.42±12.2 compared with 16.19±12.6, P>0.05).

In multivariate linear regression, there were no statistically significant interactions between treatment status with an ACE inhibitor or ARB and covariates. Covariates that were independently associated with PTSD symptoms were childhood trauma, adult trauma, being male, and being unemployed. In backward stepwise regression, β-blockers and age remained in the model, as these variables confounded the association of ACE inhibitor or ARB with PTSD symptoms. After adjusting for the above covariates, individuals treated with an ACE inhibitor or ARB had significantly decreased risk of current PTSD symptoms compared to individuals not receiving an ACE inhibitor or ARB (β=−2.83, SE=1.4, P=0.044; FIG. 5).

Frequency of childhood and adult trauma were independently associated with all the outcomes of interest; therefore, they were included in every model. Unemployment was also independently associated with hyperarousal symptoms, as assessed by the PSS, and current and lifetime PTSD symptoms, as assessed by the CAPS. After adjustment for the above covariates, individuals receiving an ACE inhibitor or ARB had a significantly decreased risk of current PTSD symptoms (β=−7.16, SE=2.78, P=0.010). Age and diuretics also remained in the model examining hyperarousal symptoms as the outcome, as they were found to be confounders. After adjusting for the above covariates, individuals taking ACE inhibitors or ARBs had a significantly decreased risk of PTSD hyperarousal symptoms (β=−1.22, SE=0.6, P=0.028; FIG. 5).

Beta-Blockers, age, and diuretics were included in the model examining lifetime PTSD symptoms as the outcome, as they were found to be confounders. After adjustment for the above covariates, individuals taking an ACE inhibitor or ARB had a significantly decreased risk of lifetime PTSD symptoms (β=−1.22, SE=0.56, P=0.028; FIG. 5).

Age, β blockers, other blood pressure medications, and income level all remained in the model examining avoidance/numbing symptoms as the outcome, as they were found to be confounders. After adjustment for the above covariates, the effect of taking an ACE inhibitor or ARB remained insignificant.

Lastly, being male was independently associated with intrusive symptoms and remained in the model. β-Blockers and age were also included, as their presence confounded the effect of ACE inhibitors or ARBs on intrusive symptoms. After adjustment for the above covariates, individuals treated with an ACE inhibitor or ARB had a significantly decreased risk of PTSD intrusive thoughts symptoms (β=−1.01, SE=0.5, P=0.029; Table 4).

### Measurements

Trauma exposure was measured using the TEI, a 14-item screening instrument for lifetime history of traumatic events. For each traumatic event, experiencing and witnessing of the event is assessed separately. The TEI also assesses frequency of trauma exposure within each trauma type. Measured as a continuous variable, frequency of exposure to traumatic events was used as a potential covariate. As previous studies have shown associations between chronic stress and blood pressure, there may be an indirect association between taking a blood pressure medication and chronic stress, which may be partly measured by frequency of traumatic events.

The primary outcome of interest in this study was PTSD symptom severity; therefore the principal measurement used for analysis was the PSS, a psychometrically valid 17-item self-report scale that measures PTSD symptom severity during the 2-week period immediately prior to study assessment. See Schwartz et al. Pain medication use among patients with posttraumatic stress disorder. Psychosomatics. 2006. 47(2):136-142. See also Schwartz et al., Psychiatr Serv, 2005, 56(2):212-215, Foa & Tolin, Trauma Stress, 2000, 13(2):181-191, and Coffey et al. Trauma Stress, 1998, 11(2):393-399.

PTSD Symptom Scale frequency items (measured as “0: not at all” to “3: 5 or more times a week) were summed to obtain a continuous measure of PTSD symptom severity. The major subtypes of posttraumatic stress symptoms were examined, including hyperarousal symptoms, avoidance or numbing symptoms, and intrusive thoughts, using the symptom-specific subscales of the PSS. Foa & Tolin, Comparison of the PTSD Symptom Scale-Interview Version and the Clinician-Administered PTSD scale, Trauma Stress, 2000, 13(2):181-191. The categorical diagnosis of PTSD was initially determined based on Diagnostic and Statistical Manual of Mental Disorders, Fourth Edition (DSM-IV) A-E criterion responses to the PSS questionnaire.

Additionally, information from the Clinician Administered PTSD Scale (CAPS) was also used to examine the effect of ACE inhibitors or ARBs on the severity of both current and lifetime PTSD symptoms. An interviewer-administered diagnostic instrument with excellent psychometric properties, the CAPS uses DSM-IV scoring criteria to generate a categorical diagnosis of PTSD, as well as a continuous measure of the extent and severity of lifetime and current posttraumatic stress symptoms.

While both the PSS and CAPS can generate symptoms to assess PTSD severity, CAPS adds additional information about lifetime PTSD symptoms. For each of the 17 diagnostic criteria, the CAPS rates frequency and intensity scores on a scale of 0 (absent) to 5 (extremely severe). This analysis used the CAPS to obtain both continuous lifetime and current PTSD variables (scores from 0 to 170).

The primary exposure of interest in this study, collected by physicians based on participants' reports, was whether an individual was prescribed an ACE inhibitor or ARB. The data on ACE inhibitors and ARBs were pooled since there were a small number of individuals taking ARBs (n=17 or 3.16% of the sample) and because of similar mechanisms of action.

Potential covariates assessed in the analysis included other blood pressure medications (categorized into β-blockers, calcium channel blockers [CCBs], diuretics, and other for medications with another mechanism of action), whether an individual was currently taking a psychiatric medication, current substance abuse, body mass index (BMI), frequency of adult trauma (as assessed by the TEI), and childhood trauma. Childhood trauma was assessed using the CTQ, a self-report inventory assessing 3 types of childhood abuse: sexual, physical, and emotional. Studies have established internal consistency, stability overtime, and criterion validity of both the original 70-item CTQ and the current brief version.

Demographic information assessed as potential covariates included: sex, age, current employment, household income level ($0-249, $250-499, $500-999, $1,000-1,999, or greater than $2,000 per month), education (categorized into less than 12^{th} grade, high school graduate or GED, some college or technical school, or college graduate and higher education), and race (dichotomized into “African American” and “other;’ due to the small number of non-African American subjects in the analysis).

Missing data included information on race (n=3 or 0.6% of the sample), income (n=12 or 2.4% of the sample), employment (n=3 or 0.6% of the sample), education (n=3 or 0.6% of the sample), current psychiatric medications (n=247 or 48.9% of the sample), current substance abuse (n=9 or 1.8% of the sample), adult traumatic experiences (n=6 or 1.2% of the sample), and childhood traumatic experiences (n=19 or 3.8% of the sample). Because close to half of the sample had missing data on current psychiatric medication and BMI (n=247 or 48.9% of the sample and n=244 or 48.3% of the sample, respectively), these variables were excluded as potential covariates from modeling analysis.

### Analysis

Analysis was performed using SAS 9.2 (SAS Institute Inc, Cary, N.C.) statistical software. To statistically evaluate the independent effect of ACE inhibitor and ARB medication on PTSD symptom severity among patients exposed to trauma, linear regression models were fit with PTSD symptoms, measured by the total PSS score as the continuous outcome variable. Two-way multiplicative interaction between the categorical variable of active treatment with an ACE inhibitor or ARB medication and other covariates were assessed in a full model. Potential confounders were assessed using multiple approaches. First, directed acyclic graphs were constructed using information from previous literature. Second, a 2-table approach was used, in which the association of each potential confounder was examined in relation to both PTSD symptoms and active treatment with an ACE inhibitor or ARB. Finally, a backward regression modeling approach was used; in which variables were removed 1 at a time and assessed for statistical significance and effect on the estimate for the main exposure of interest.

Descriptive analysis of the variables was performed, stratified by categorical PTSD diagnosis. X² tests were used to assess the association of PTSD diagnosis with ACE inhibitors or ARBs, 13-blockers, CCBs, diuretics, sex, race, income, employment, education, current psychiatric medications, and current substance abuse. Two sampled t tests were used to assess the association of PTSD diagnosis with BMI, age, and adult and childhood trauma (as assessed by the TEI and CTQ, respectively).

To evaluate the effect of different categories of blood pressure medications, including ACE inhibitors and ARBs, on PTSD symptoms, univariate analysis of variance was performed. To statistically evaluate the independent effect of ACE inhibitors and ARBs on PTSD symptoms, multivariable linear regression models were constructed, using potential confounders, which were previously identified. Collinearity between the covariates was assessed and linear regression assumptions were checked. To analyze the effect of treatment with different types of blood pressure medications on the severity of PTSD symptom subtypes, multivariable linear regression models were created and tested using continuously scaled PTSD symptoms among each subtype as the outcome. A P value of less than 0.05 was considered statistically significant for analysis.

### Effect of Administration of Losartan on Learned Fear

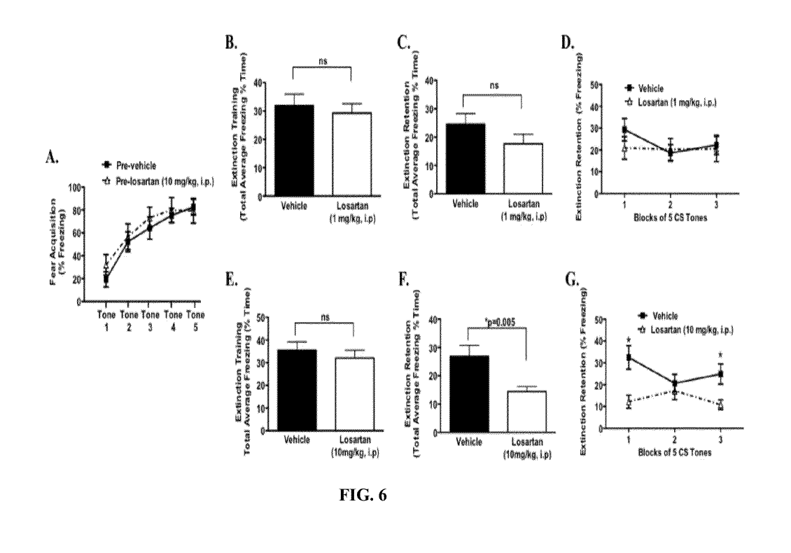

To further understand the mechanism by which angiotensin II blockers reduce PTSD symptoms, the effects of the selective AT1R antagonist losartan were examined in an animal model of PTSD-like symptoms. Animals were cue-fear trained using Pavlovian fear conditioning consisting of five pairings of a conditioned stimulus (CS) tone (12 kHz) co-terminating with a shock or unconditioned stimulus (US). In the absence of drug, both groups of animals exhibited normal acquisition of fear as determined by percent freezing to the five tone-shock or CS-US pairings (FIG. 6A). Twenty-four hours later, the effects of losartan were examined on fear expression (also considered the extinction training session) to the presentation of 15 trials of CS cues in a different context. Groups were given either losartan (1 mg/kg or 10 mg/kg i.p.) or saline 40 minutes prior to fear expression/extinction training. During extinction training, at 1 mg/kg losartan, there was no difference in total average freezing (FIG. 6B). Extinction retention testing in these animals was performed 24 hours later in the absence of drug and, as shown in FIG. 6C-D, there were no freezing differences between groups. Therefore in a separate group of animals, a higher dose of losartan (10 mg/kg i.p.) was examined. As shown in FIG. 6E, during extinction training/fear expression there were no differences between groups in total average freezing. Twenty-four hours later in the absence of drug, long-term fear memory was assessed (extinction retention). As shown in FIG. 1F, total overall freezing was significantly reduced in these mice (t(48)=2.9*P<0.01). Moreover, as determined by repeated-measures 2 way ANOVA, there was a significant main effect for treatment in the losartan group (10 mg/kg i.p), which exhibited significantly less freezing to CS presentation during extinction retention testing (F_{1,144}=14.6; *P<0.001) (FIG. 6G). Bonferroni post hoc analysis revealed significant reductions in freezing during the first and third blocks of five CS tone presentations (*P<0.05) (FIG. 6G). Taken together, these data indicate that losartan does not affect fear expression, but enhances retention of fear extinction, suggesting that AT1R antagonism may reduce fear memory through enhancing the consolidation of extinction learning.

### No Blood Pressure or Anxiety-Like Effects Following Acute Administration of Losartan

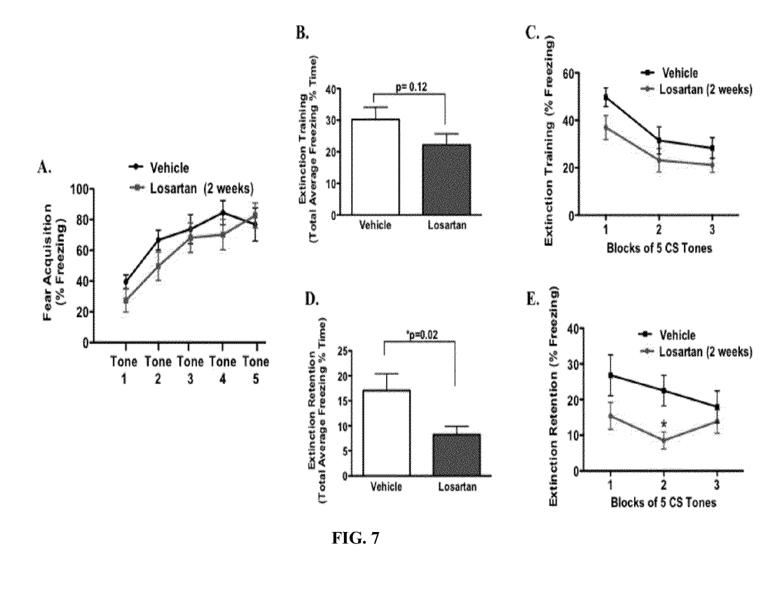

In order to determine whether inhibition of the renin-angiotensin system is involved in modulating fear directly or indirectly, both acute and chronic inhibition of the angiotensin type 1 receptor (AT1R) were examined using the AT1R antagonist losartan (1 mg/kg and 10 mg/kg). One possible hypothesis is that the above effects on extinction consolidation were due simply to compensatory changes following acutely lowered blood pressure or anxiety level. A dose of 10 mg/kg is commonly used in rodents, however the effects on baseline blood pressure are mixed. When given acutely or chronically, some studies show reductions in baseline blood pressure while others show no effect. In the current study acute administration of losartan (i.p. 10 mg/kg) had no effect on baseline blood pressure (FIG. 7A).

To determine whether losartan affects baseline levels of generalized anxiety, animals were tested in the elevated plus and open field mazes. No differences in anxiety-like behavior were found between animals treated with losartan (i.p. 40 minutes prior to test) compared to saline treated as measured by distance traveled and time spent in open arms (FIG. 7B-C). These data suggest that acute administration of losartan at 10 mg/kg i.p does not affect baseline levels of blood pressure or measures of anxiety-like behavior in these animals.

### Effect of Chronic Losartan Treatment on Learned Fear and Neuroendocrine Measures

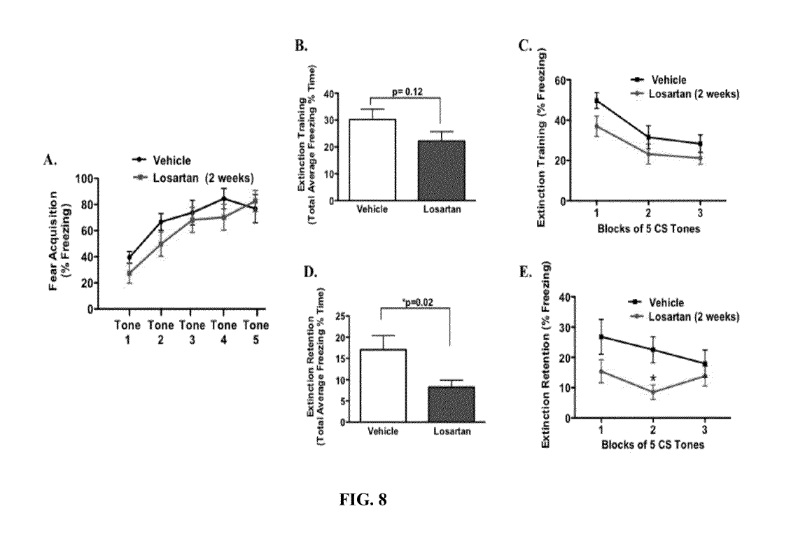

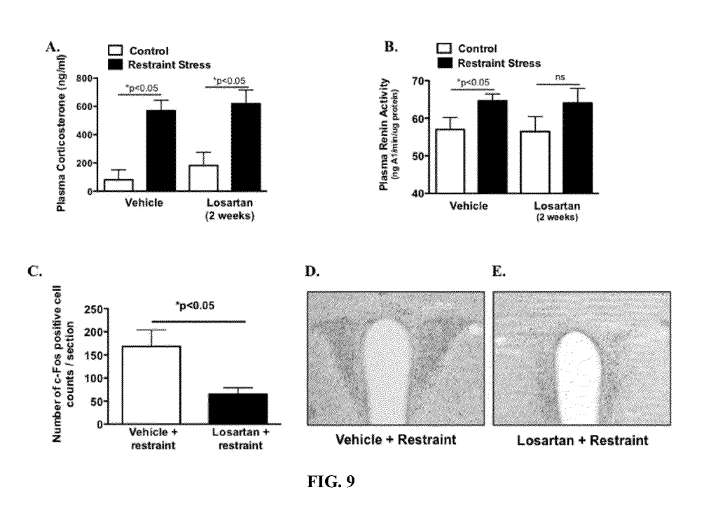

The chronic effects of AT1 R antagonism on the extinction of fear to were examined. Two-week osmotic mini-pumps were implanted and losartan (10 mg/kg/day) was subcutaneously infused. The dose of 10 mg/kg/day was chosen based on the acute study showing enhanced extinction retention at this dose. On day 10, animals underwent Pavlovian fear conditioning as described above. As shown in FIG. 8A, there was no difference in the acquisition of fear as determined by percent freezing in mice treated with losartan compared to vehicle. Furthermore, when fear expression was evaluated in these mice 24 hrs later, despite the decreased trend, there was no significant difference in total average freezing or when expressed in blocks of 5 CS trials (FIG. 8B-C). Consistent with the effects of acute administration of losartan on extinction, mice treated chronically with losartan displayed enhanced extinction retention of fear memory when expressed over the total average freezing period (FIG. 8D, t(35)=2.4*P<0.05). Repeated-measures ANOVA, during extinction retention testing, revealed a significant main effect for treatment in the losartan group (10 mg/kg/day) as they exhibited significantly less freezing to CS presentation (F1,108=8.8; *P<0.005) (FIG. 8E). Bonferroni post hoc analysis revealed significant reductions in freezing during the second block of five CS tone presentations (FIG. 8E, *P<0.05). Similar to the acute administration, there were no differences between groups in generalized anxiety testing as determined by percent time in center and distance traveled during the open field test or baseline blood pressure. These data provide evidence that long-term AT1R inhibition is involved in fear memory, independent of any indirect effects on blood pressure or measures of baseline anxiety.

The hypothalamic pituitary axis (HPA) stress axis could influence the level of learned fear. In order to examine the chronic effects of losartan on the HPA, stress response was examined as well as plasma renin, a measure of endogenous angiotensin II activity. Animals were exposed to 30 minutes of restraint stress and plasma levels of corticosterone and renin measured. Significant elevations in corticosterone levels were observed following restraint stress but these levels were unaltered by losartan (F1,35=193.4; *P<0.0001) (FIG. 9A).

Similarly plasma renin activity was elevated following restraint and this was unchanged by losartan (FIG. 9B, t(17)=2.1*P<0.05). As an index of central HPA neuronal activation, c-Fos activation was then examined in the paraventricular nucleus (PVN). As shown in FIG. 9C, losartan treated animals exhibited reduced c-Fos activation in the PVN (t(10)=2.3*P<0.05). These data suggest that chronic AT1R inhibition does not prevent elevations in peripheral indices of the stress response but may influence central stress activation sites in the brain such as the PVN, which may influence other neuronal circuits involved in the extinction of fear.

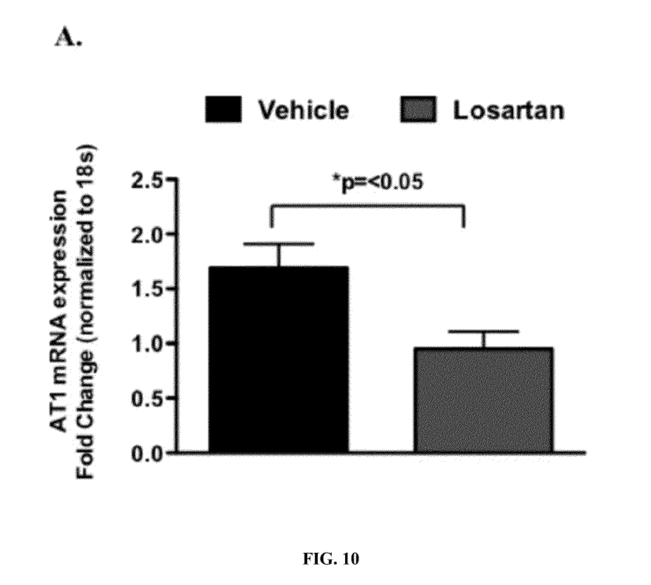

In order to examine whether chronic losartan influences AT1 receptor expression in the amygdala, a key limbic brain structure in both the acquisition and extinction of fear was studied. As shown in FIG. 10, following extinction training, chronic losartan treatment significantly reduced amygdala AT1R mRNA expression compared to the vehicle group (t(11)=2.8*P<0.05). It is possible this reduction in amygdala AT1R may be downstream of chronic AT1R inhibition, thus underlying the enhanced extinction of fear memory that was observed in the losartan treated animals.

What is claimed:

1. A method of treating or preventing posttraumatic stress disorder comprising of administering a pharmaceutical composition comprising effective amount of an angiotensin-converting enzyme inhibitor to a subject in need thereof.

2. The method of claim 1, wherein the angiotensin-converting enzyme inhibitor is administered in combination with an angiotensin receptor blocker.

3. The method of claim 1, wherein the subject is at risk of, exhibiting symptoms of, or diagnosed with posttraumatic stress disorder.

4. The method of claim 1, wherein the angiotensin-converting enzyme inhibitor is selected from perindopril, captopril, enalapril, eprosartan, lisinopril, quinapril, benazepril, imidapril, trandolapril, fosinopril, ramipril, and zofenopril.

5. The method of claim 2, wherein the angiotensin receptor blocker is losartan, irbesartan, olmesartan, candesartan, valsartan, telmisartan, and azilsartan.

6. The method of claim 1, wherein the pharmaceutical composition is administered in combination with another therapeutic agent.

7. The method of claim 6, wherein the therapeutic agent is selected from propranolol, citalopram, escitalopram, fluoxetine, fluvoxamine, paroxetine, sertraline, prazosin, clonidine, carbamazepine, topiramate, zolpidem, lamotrigine, valproic acid, lithium carbonate, buspirone, risperidone, cyproheptadine, nefazodone, trazodone, amitriptyline, imipramine, phenelzine, or corticosterone and combinations thereof.

8. A method of treating or preventing posttraumatic stress disorder comprising administering a pharmaceutical composition comprising an effective amount of an angiotensin receptor blocker to a subject in need thereof.

9. The method of claim 8, wherein the subject is at risk of, exhibiting symptoms of, or diagnosed with post-traumatic stress disorder.

10. The method of claim 8, wherein the angiotensin receptor blocker is losartan, irbesartan, olmesartan, candesartan, valsartan, telmisartan, and azilsartan.

11. The method of claim 8, wherein the pharmaceutical composition is administered in combination with another therapeutic agent.

12. The method of claim 6, wherein the therapeutic agent is selected from propranolol, citalopram, escitalopram, fluoxetine, fluvoxamine, paroxetine, sertraline, prazosin, clonidine, carbamazepine, topiramate, zolpidem, lamotrigine, valproic acid, lithium carbonate, buspirone, risperidone, cyproheptadine, nefazodone, trazodone, amitriptyline, imipramine, phenelzine, or corticosterone and combinations thereof.
